SEQUENCE LISTING (12 sequences)

SEQ ID NO: 1 - DNA, 186 bp, synthetic construct
  source 1..186: mol_type = other DNA
atgccccagcttatattcctgctgctgatcatcgccgccgcgtggtacggctaccgctcgttcaagcgtgaggcggcgcgcgtttcgcagcgtgtccggcaggccgaaaaggaagcacagaaccgtgcccatggcacgctggtgcaagatcccaagaccggcgaatattacgtccgcaaggattga

SEQ ID NO: 2 - DNA, 1814 bp, synthetic construct
  source 1..1814: mol_type = other DNA
atgctcgatagcctgcgttccgccgctcaatcctggatcgccaaacttttgcttggcctgcttgtgctaagctttgcgatctggggcatctcggatgtcttccgtggcggcatggcaggcaacgccgcgctgacagccggtgattccgaggtcagcgcgaccgattatcgctttgcctacgaacagcagcttatgcgcctttcgcagcagttccgccagcggctcacccgcgaacaggccaaggcaatcggcgtcgagaaccaggttctggcccagcttgccgccggtgtcgtgctggatgaagcagcccgcaacatgcagcttggcctttcccaggacgccattgcccgcctgaccgccgaagatcccgccttccatgatgcaggcggcaatttcaaccgcgcgcggtttgatgcggttttgcgccagtccggcattcgcgccgaggattatctggaaaaccgtgcgaaagtcgcccgccgccagcagatcgtcgaagcagcaacggacggcatgaaactgccggatacgatgctgaaggcacttgccctctatcagggcgaaagccgcagcgccgactatatcgatgtgcctgccgaaaaggccgacgccattcccgcaccgtcagaggatgtgctcaaggcctattttgaagcgcacaaggaagattacaaggcgccggaatatcgcaagatcagctatgtgaagctggagccgtccgatattgccgaccctgccgccgtcacgcaggatgagatcagcgaatattatgaaaagaacaaggcccgtttcagcacgcctaagacgcgcaccatcgaacagctgaactttgccgacgatgccgccgctgaagccgcccaccagaagatcgaggcaggcaccagcttcctcgatatcggcaaggaacagggcaagaccgaaagcgatctgacgctcggcacttttgaaaaatccggcattcccgaccagaccattgccgacgccgccttcgcgcttggcgaaggtgcggtcagcccggttgtcaagggaatcttcggcccggtcctgctgcgtgtaacgaaaatcaacccggcgcatgagaagccgctttccgaagtcgaaggagaaatccgcaacacgctcgccaccaatattgcagccagcagcatcaacagcctgcacgattcctatgagcaggaacgttcggacggggcaaccatggccgatgtcgccaaaaagctgcacctgaaaatggtaacggtcgatgcaatcgatgccgaaggcgacgatcctgcgggcaagcctgttgaactgccgaatgcgcaggcgctgcttccagccgtctttcaggctgaacagggcttcgacaacgacccgctcaccatgggtaatatcggctatctctggtatcaggtggatggcgttacccctgcccgcgaccgcacgctcgatgaagtcaaggacaaggtcgtggcggcatggaagggcgaggaagccgtcaagcgcctcaaccagcgcatggaagaattgaagaagcgcgtcgaagacggcgcaacgcttgacacaatcgcttccgaactgggccttgaaaagcagaccaagcgcggcatcacccgcaccaccaacgacgccgacatcggttcggctgcaaccgcgcagattttccgtggcgcgaacggtctggtcggcacggctgccgcgccctccgacgatgcgcaaattctcttcaaggtaacggaggtcaccaaacctgccgccgccggaccggaagcaatcccggaagatcagcagaaatatctggcgaccgcgcttgcggatgatatgctgcaacagct

SEQ ID NO: 3 - DNA, 534 bp, synthetic construct
  source 1..534: mol_type = other DNA
ttgaaaacggctatcgaccgtggcggttcgttcaaggccgagcttgatacctatgtctcggtggcgccgcaggatgcttccgtggaagggctgcgcgcctatgcggacaagggcgtgccgacaattgccgatctaaatgcatggttcggtgcggttgccaatcgcatcgtggccacggaaaacaagcttcccgccgatgccagcctatgggatcagttgctggcgagcgccagggggctggtcagcgtcaggcccgttgcgggcaatgtaagcggcaccggcgtcgggccgacgacggcgcgcatggaggcggcattacaggcaggcgatcttgaccgggccatcggcgaatgggagcaattgccagccgatgcgaaagcggtttccgaagaatttgccagccagatgaaggcgcgccgcgatgccgatgcgctggtgcagcgtcttgtggccgatagcctcaagccgaaagcgccggtgacgggtgcttccgataatacaaccggcgaggctccggcctcttccggtcagtaa

SEQ ID NO: 4 - DNA, 36 bp, synthetic construct
  source 1..36: mol_type = other DNA
agccggtacgatcttgccgtctttttctcccggagc

SEQ ID NO: 5 - DNA, 25 bp, synthetic construct
  source 1..25: mol_type = other DNA
aagcttatgtctcgttacgatctgg

SEQ ID NO: 6 - DNA, 25 bp, synthetic construct
  source 1..25: mol_type = other DNA
catatgctgtacgacgcccatctct

SEQ ID NO: 7 - DNA, 30 bp, synthetic construct
  source 1..30: mol_type = other DNA
aagcttatgtcccgttacgatctggctgtt

SEQ ID NO: 8 - DNA, 26 bp, synthetic construct
  source 1..26: mol_type = other DNA
catatgttttttatcagcggcctgtt

SEQ ID NO: 9 - DNA, 26 bp, synthetic construct
  source 1..26: mol_type = other DNA
aagcttatgtctcgctacgacctggc

SEQ ID NO: 10 - DNA, 26 bp, synthetic construct
  source 1..26: mol_type = other DNA
catatgggagctgccaccggtaatca

SEQ ID NO: 11 - protein, 12 aa, synthetic construct
APGEKDGKIVPA

SEQ ID NO: 12 - DNA, 36 bp, synthetic construct; identical to SEQ ID NO: 4
  source 1..36: mol_type = other DNA

(19) United States
(12) Patent Application Publication
(10) Pub. No.: US 2025/0298035 A1
(43) Pub. Date: Sep. 25, 2025

(54) BRUCELLOSIS CELL IMMUNE PROTEIN AND USE THEREOF

(71) Applicant: TECON BIOPHARMACEUTICAL CO., LTD, Urumqi, Xinjiang (CN)

(72) Inventors: Sun He, Urumqi, Xinjiang (CN); Chuanyu He, Urumqi, Xinjiang (CN)

(21) Appl. No.: 18/863,698

(22) PCT Filed: Dec. 18, 2023
(86) PCT No.: PCT/CN2023/139476

§ 371 Date: Nov. 7, 2024

(30) Foreign Application Priority Data
Feb. 22, 2023 (CN) 202310147968.6

(51) Int. Cl. G01N 33/68; C07K 14/23; C12N 15/70; G01N 33/58

(52) U.S. Cl. CPC G01N 33/6872; C07K 14/23; C12N 15/70; G01N 33/581; C07K 2319/21; G01N 2333/54

(57) ABSTRACT

The present invention provides a Brucellosis cell immune protein and use thereof. In the present invention, firstly, a Brucellosis cell immune protein with relatively high immunogenicity is screened by means of antibody spectrum technology; then, the protein gene sequence of Brucella are subjected to fusion expression by using molecular biology technology, so as to improve the ability for producing an immune response which is induced by an antigen; next, the antigen is expressed in vitro and then subjected to multi-stage purification, and the obtained purified protein is used as a stimulant; and finally, anti-coagulated blood from an immunized animal is collected, the stimulant is added thereto, the mixture is incubated for 24 hours at 37° C., the resulting product is centrifuged to collect a supernatant, and the level of IL-17 in the supernatant is detected by an ELISA method.

### CROSS-REFERENCE TO RELATED APPLICATIONS

This application is a United States National Stage Application filed under 35 U.S.C. § 371 of PCT Patent Application Serial No. PCT/CN2023/139476, filed on Dec. 18, 2023, which claims the priority of Chinese Patent Applications 202310147968.6, entitled “Brucellosis cell immune protein and use thereof” and filed on Feb. 22, 2023, the entirety of which is incorporated herein by reference.

### REFERENCE TO SEQUENCE LISTING

The Substitute Sequence Listing XML file is submitted to replace the original sequence listing filed in the PCT Application No. PCT/CN2023/139476, with a file name of “Substitute_Sequence_Listing.xml”, a creation date of Nov. 7, 2024, and a size of 14,129 bytes. The Substitute Sequence Listing XML file is a part of the specification and is incorporated in its entirety by reference herein.

### FIELD OF THE INVENTION

The present invention belongs to the field of microbial gene engineering, and specifically relates to a Brucellosis cell immune protein and use thereof.

### BACKGROUND OF THE INVENTION

Brucellosis (Brucella disease) is a zoonotic disease that is caused by various Brucella bacteria, which has a significant impact on human health and economic returns of animals. As with other infectious diseases, vaccine immunization is the most effective strategy of Brucella disease preventing and controlling, and the currently-used vaccines include A19, S2, M5, A19-ΔVirB12 and the like. A protective efficacy can be effectively provided after vaccinated, but the protection efficacy of vaccine after inoculation will gradually decrease with the extension of time.

In order to timely detect the protective efficacy of the vaccine, in the prior art, a method combining immunization protective test and antibody detection is usually adopted, wherein, Rose bengal plate agglutination test (RBT) and Standard-tube agglutination test (SAT) detection methods are usually adopted. But the antibody detection cannot directly reflect the protective efficacy change of the vaccine, because the Brucella belongs to intracellular parasitic bacteria, such that body prophylaxis and removal of Brucella are mainly achieved via cellular immunity routes, therefor the cellular immunity is a main index of the protective efficacy provided by Brucella vaccine, and at present, only a immunization protective test can be used for efficacy assessment after vaccine immunization, that is, virulent strains are used for challenge after vaccine immunization, and the efficacy of the vaccine is evaluated by assessing a bacteria loading capacity in the tissues which are subjected to challenge. This method is complex to operate, needs to be carried out in a biological safety protection three-stage laboratory, and is high in economic investment. Meanwhile, the operation of the virulent strains is at a high biological safety risk and there is a possibility of human infection. At present, there is no detection method capable of quickly, simply and effectively assessing the cellular immune level after vaccine immunization.

### SUMMARY OF THE INVENTION

The technical problem to be solved by the present invention is to provide a reagent and method for effectively detecting immunoprotection efficacy by detecting the level of cytokine IL-17 in whole blood of animals after immunization.

In order to solve the technical problem above, the present invention provides a Brucellosis cell immune protein.

The present invention further provides a use of the Brucellosis cell immune protein in preparation of a reagent for detecting an immune level of a Brucella vaccine and a use of the Brucellosis cell immune protein in detecting the immune level of the Brucella vaccine.

The technical solutions adopted by the present invention are as follows:

A Brucellosis cell immune protein, wherein the Brucellosis cell immune protein is any one of three antigens: BMEI1536*, BMEI0845* and BMEI0178*;
wherein, the BMEI1536* has a nucleotide sequence as shown in SEQ ID No.1;
wherein, the BMEI0845* has a nucleotide sequence as shown in SEQ ID No.2;
wherein, the BMEI0178* has a nucleotide sequence as shown in SEQ ID No.3.

Preferably, the Brucellosis cell immune protein is BMEI1536*.

The above Brucellosis cell immune protein is specifically obtained by expressing a fusion protein sequence of a Brucella T cell epitope peptide fragment with antigen gene sequences of BMEI1536, BMEI0845 and BMEI0178 respectively, followed by recombinant transformation, incubation expression, and purification.
wherein, the Brucella T cell epitope peptide fragment is APGEKDGKIVPA, SEQ ID NO. 11, and has a nucleotide sequence thereof as shown in SEQ ID No. 4.
wherein, the recombinant transformation comprising synthesizing the sequence which is referred as to the fusion protein sequence into an expression vector pET-28a to obtain a recombinant plasmid, and then transforming the recombinant plasmid into a host strain BL21.
wherein, the incubation expression is performed under the following conditions: at 37° C. and 200 r/min for 12-16 h.
wherein, the purification is nickel column purification and molecular sieve purification.

A reagent for detecting an immune level of a Brucella vaccine comprising the above-described Brucellosis cell immune protein also falls within the protection scope of the present invention.
wherein, the reagent is any one of BMEI1536*, BMEI0845* and BMEI0178*, preferably BMEI1536*.

A method of detecting an immune level of a Brucella vaccine using the above-described Brucellosis cell immune protein also falls within the protection scope of the present invention,
wherein, specific detection steps comprise: collecting a blood sample of a calf after immunization, adding the Brucellosis cell immune protein thereto, collecting a supernatant after incubation, and detecting a concentration of IL-17 in the supernatant by an ELISA method;
wherein, the Brucellosis cell immune protein has a concentration of 600-1500 μg/ml; and the incubation is performed under the following conditions: at 37° C. for 16-48 h.

Preferably, when the Brucellosis cell immune protein is BMEI1536*, the antigen protein concentration thereof is 800-1200 μg/ml, the incubation time is 32-48 h, and at this time, the concentration of IL-17 may reach 91.029-174.898 pg/ml. When the Brucellosis cell immune protein is BMEI0845*, the antigen protein concentration thereof is 1,000-1,500 μg/ml, the incubation time is 24-40 h, and at this time, the concentration of IL-17 may reach 95.976-128.955 pg/ml. When the Brucellosis cell immune protein is BMEI0178*, the antigen protein concentration thereof is 800-1200 μg/ml, the incubation time is 40-48 h, and at this time, the concentration of IL-17 may reach 103.308-146.181 pg/ml.

Preferably, when the Brucellosis vaccine cell immune protein is BMEI1536*, the antigen protein concentration thereof is 800-1200 μg/ml, the incubation time is 32-48 h, and at this time, the concentration of IL-17 may reach 91.029-174.898 pg/ml.

Most preferably, when the Brucellosis vaccine cell immune protein is BMEI1536*, the antigen protein concentration thereof is 1000 μg/ml, the incubation time is 40 h, and at this time, the concentration of IL-17 may reach 174.898 pg/ml.

The present invention has the following beneficial effects:
(1) In the present invention, the immune effect on the Brucella is detected by adopting a method for detecting the concentration level of IL-17 for the first time, and this method is simple to operate, with high safety, and is accurate and rapid.
(2) In the present invention, the Brucellosis cell immune protein capable of effectively stimulating an immune response in vitro is provided, and a fusion expression of Brucella T cell epitope and antigen gene sequence is carried out, so that the ability of generating immune response inducted by antigen is further improved.

### BRIEF DESCRIPTION OF THE DRAWINGS

The present invention will be further illustrated in detail below in combination with the accompanying drawings and specific embodiments, and the above and/or other advantages of the present invention will become more clear.

FIG. 1 shows the cloning result of a recombinant gene. Note: M: D2000; 1: BMEI0845; 2: BMEI1536; 3: BMEI0178.

FIG. 2 shows the verification result of double enzyme digestion of a recombinant plasmid. Note: M1: 15000 maker; M2: D2000; 1: BMEI0845; 2: BMEI1536; 3: BMEI0178; 4: pET-28a.

### DETAILED DESCRIPTION OF THE EMBODIMENTS

Unless otherwise specially stated, the experimental methods described in the following examples are all conventional methods; and unless otherwise specially stated, the reagents and materials are all commercially available.

The experimental materials used in the following examples are as follows:
1. Experimental animal: 20 healthy calves with negative Brucella antibody, purchased from Xinjiang Xinglongsheng Livestock Technology Co., Ltd.
2. Strains: Brucella ovis M28 strains, purchased from China Veterinary Microbiological Culture Collection Center; Escherichia coli BL21 strains, purchased from Sangon Biotech (Shanghai) Inc.
3. Vaccine: Brucellosis live vaccine (strain A19), produced by Tiankang Biopharmaceutical Co., Ltd.
4. Plasmid: pET-28a plasmid, purchased from Sangon Biotech (Shanghai) Inc.
5 Experimental Reagents
(1) PBS (phosphate buffered solution) buffer dry powder, with an item No. of P1010, purchased from Beijing Solarbio Science & Technology Co., Ltd.
(2) Kanamycin, purchased from sigma.
(3) Gram stain solution, Koster's stain solution, hydrogen sulfide biochemical medium, basic fuchsin and thionine, all of which are purchased from Hangzhou TIANHE Microbial Reagent Co., Ltd.
(4) Plasmid extraction kit, nucleic acid gel recovery kit, PCR mix, T4 ligase, and ployHis tag detection antibody, all of which are purchased from Takara (Dalian) Co., Ltd.
(5) Trypticase soy broth (TSB), with an item No. of 211825; Trypticase soy agar (TSA), with an item No. of 236950; and LB liquid medium, with an item No. of 211327, all of which are purchased from BD, USA.
(6) IPTG, purchased from invitrogen.
(7) Imidazole, purchased from sigma.
(8) Bovine IL-17A ELISA kit, C-terminal polyHis and N-terminal polyHA tag detection antibodies, all of which are purchased from abcam.
6. Experimental consumables

TABLE 1
Experimental consumables

| Name of consumables | Brand |
|---|---|
| 10 μL Tip | Axygen |
| 200 μL Tip | Axygen |
| 1000 μL Tip | Axygen |
| 10 mL Pipette | NUNC |
| 20 mL Pipette | NUNC |
| 1.5 mL Centrifuge tube | Axygen |
| 2 mL Centrifuge tube | Axygen |
| 15 mL Centrifuge tube | Axygen |
| 50 mL Centrifuge tube | Axygen |
| Disposable coating rod | Biosharp |
| 0.1 cm Electric rotating cup | Bio-Rad |
| Disposable 90 culture dish | Domestic |

### Example 1

### 1. Construction of Brucella Genome ORF Recombinant Expression Library

Gene ORF expressing cloning libraries were constructed by using a high-throughput polymerase chain reaction/recombinant cloning method, by taking a Brucella genome sequence as a template. By means of high-throughput homologous recombination, the ORF sequence was cloned into a plasmid-expressing vector pET-28a to obtain a recombinant expression library.

### 2. Establishment of Protein Chip Method

### 2.1. Preparation of Chip Protein

All proteins encoded by the ORF plasmid were expressed by using a cell-free in-vitro expression system, to yield 3164 expression products in total, and each protein was separately transferred onto a customized nitrocellulose microarray slide. Each batch of transferred protein chip slides were subjected to a quality control test by automatic scanning, to check spot deposition and morphology. Protein expression conditions were detected by using antibodies against C-terminal polyHis and N-terminal polyHA tags, expression conditions of recombinant proteins were analyzed, and unexpressed proteins were not subjected to subsequent analysis.

### 2.2. Establishment of Chip Detection Method

Anti-IgG antibodies were labeled with CY5 to prepare fluorescence-labeled secondary antibodies, and optimal working conditions of primary antibodies and secondary antibodies were obtained through optimization of serum concentration and secondary antibody dilution, so as to establish a chip detection method.

### 3. Immunogenic Antigen Screening

### 3.1. Serum Preparation

Ten 3-6 months-old Brucella antibody-negative calves were randomly divided into 2 groups, with 5 calves each group. The 2 groups were injected with A19 vaccine and physiological saline, respectively. In the A19 vaccine group, each calf was subcutaneously inoculated with 6.0×10¹⁰ CFUs in the neck, the calves in the control group were injected with physiological saline, and all the calves were isolated and fed under the same conditions. The serums of all the experimental calves were collected at 21 days after immunization, and placed at −80° C. to be ready for use.

### 3.2. Antigen Screening

The calf serums in the vaccine group and the calf serums in the control group were respectively reacted with the protein chip which was subjected to detection and screening, and the specific reaction conditions were as follows: a PBS buffer solution containing protein (containing 10%-50% of glycerol) was spotted on a substrate. An incubation temperature was 37° C., and an incubation time was 2 h. Then the resultant was incubated with PBS solution (containing 5% of skim milk powder) under sealing at 37° C. for 2 h, washed with the PBS buffer (containing 10% of glycerol) for 3 times (5 min for each time: standing for 2 min, and shaking for 3 min). After standing for 15 min, a fluorescence detection was performed, to assess immunogenicity intensities of the corresponding proteins according to fluorescence intensities, and the proteins with a ratio of the fluorescence intensity of the immunization group to the fluorescence intensity of the control group greater than 2 were selected as candidate antigens. And the obtained candidate proteins are shown in Table 2, which were to be subjected to a next experimental verification.

TABLE 2
Results of reaction of the serums in the immunization
group and the serums in the control group with the chip

| Antigen | Antigen information |
|---|---|
| BMEI0845 | Peptidyl-prolyl cis-trans isomerase D |
| BMEI0178 | Hypothetical protein |
| BMEI1536 | Hypothetical protein |

### 4. Construction of Protein-Expressing Strains and Protein Purification

### 4.1 Fusion Expression of Antigen

Cell epitopes were predicted by using an online tool phyre2 (http://www.sbg.bio.ic.ac.uk/phyre2/html/page.cgi?id=index). A nucleotide sequence (AGCCGGTACGATCTTGCCGTCTTTTTCTCCCGGAGC, SEQ ID NO. 12) corresponding to a predicted Brucella calf T cell epitope peptide fragment (APGEKDGKIVPA, SEQ ID NO. 11) was connected with nucleotide sequences corresponding to three screened antigens to obtain antigens BMEI1536*, BMEI0845* and BMEI0178*, the nucleotide sequences of which were shown in SEQ ID No.1, SEQ ID No. 2 and SEQ ID No. 3, respectively.

### 4.2. Construction of Immune Protein Recombinant Expression Strains

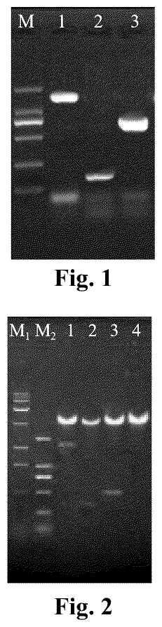

According to the sequence information of the three antigens after fusion expression in 4.1, the sequences were synthesized into an expression vector pET-28a to obtain recombinant plasmids, then the recombinant plasmids were transformed into Escherichia coli BL21, and protein recombinant expression strains were obtained by screening, which may be used for subsequent protein expression (FIG. 1 and FIG. 2).

Specifically, upstream and downstream primers were designed according to the coding region sequences of the antigens BMEI1536*, BMEI0845* and BMEI0178*, and BMEI1536*, BMEI0845* and BMEI0178* gene fragments were recovered by gel cutting.

The recovered PCR products and plasmids pET-28a were subjected to double enzyme digestion via restriction endonucleases HindIII and NdeI, and the enzyme digestion products were purified by 1% agarose gel and then recovered to be ready for use.

Primer information and PCR reaction systems are shown in Table 3 and Table 4.

TABLE 3
Primer sequence information

| Name of | | | Endo | Product size |
|---|---|---|---|---|
| antigen | | Primer sequence | nuclease | about (bp) |
| BMEI1536* | Upstream | AAGCTTATGTCTCGTTACGATCTGG | HindIII | 750 |
| | | (SEQ ID NO. 5) | | |
| | Downstream | CATATGCTGTACGACGCCCATCTCT | NdeI | |
| | | (SEQ ID NO. 6) | | |
| BMEI0845* | Upstream | AAGCTTATGTCCCGTTACGATCTGGCTGTT | HindIII | 1230 |
| | | (SEQ ID NO. 7) | | |
| | Downstream | CATATGTTTTTTATCAGCGGCCTGTT | NdeI | |
| | | (SEQ ID NO. 8) | | |
| BMEI0178* | Upstream | AAGCTTATGTCTCGCTACGACCTGGC | HindIII | 897 |
| | | (SEQ ID NO. 9) | | |
| | Downstream | CATATGGGAGCTGCCACCGGTAATCA | NdeI | |
| | | (SEQ ID NO. 10) | | |

TABLE 4
PCR system

| Reagents | Volume (μL) |
|---|---|
| Template | 1.5 |
| 2 × TransStart Mix | 15 |
| Upstream primer | 1 |
| Downstream primer | 1 |
| ddH₂O | 11.5 |
| In total | 30 |

A recombinant plasmid construction and transformation method was as follows:
(1) 2 μL of linearized pET-28a vectors, 1 μL of antigen fragments, 5 μL of 2×ClonExpress Mix, 2 μL of ddH₂O were gently sucked with a pipette and uniformly mixed by flicking, and the obtained reaction solution was collected at the bottom of a tube by short-time centrifugation;
(2) The resultant was heated in water bath at 50° C. for 5 min, and immediately placed on ice for cooling;
(3) Competent cells BL21 were thawed on ice;
(4) 10 μL of the recombinant products were added to 100 μL of competent cells BL21, the tube wall was gently flicked for uniform mixing, and the mixture was allowed to stand on ice for 30 min;
(5) After being subjected to heat shock in a water bath at 42° C. for 45 s, the resultant was immediately placed on ice to cool for 2-3 min;
(6) 900 μL of antibiotic-free LB liquid medium was added, and the resultant was cultured at 37° C., 200 rpm for 1 h;
(7) A Kanamycin resistance LB solid medium plate containing Kanamycin at a concentration of 100 μg/ml was preheated in an incubator at 37° C.;
(8) The resultant was centrifuged at 5000 r/min for 5 min, 900 μL of supernatant was discarded, the bacteria system was suspended with the remaining medium, and gently and uniformly coated on the plate containing Kanamycin resistance with a sterile coating rod; and
(9) The resultant was inversely cultured in the incubator at 37° C. and 200 r/min for 12-16 h.

### 4.3. Purification of Immune Protein Recombinant Expression Strains

An LB culture medium containing 1% e kanamycin was prepared, and inoculated with recombinant expression strains prepared in 4.2 at a ratio of 1:100, the resultant was cultured at 37° C. and 200 r/min for 4 h; then an inducer (IPTG) was added at a ratio of 1:1000, and the resultant was cultured in a shaker at 37° C. and 200 r/min overnight to a large scale; then the obtained bacteria solution was centrifuged at 8000 rpm for 15 min, the supernatant was discarded, and the resultant was resuspended and washed with PBS for three times; the obtained precipitate was resuspended with 300 mL of protein purification A liquid, and the resuspended bacteria solution was crushed on an ultrasonic crusher for 2 times (15 min for each time) until the bacteria solution was clear; and antigen proteins were collected, and subjected to nickel column purification and molecular sieve purification, to obtain purified products of three antigen proteins, respectively. Protein concentration was detected by using a BCA protein quantitation kit.

Specifically, protein purification steps were as follows:
(1) A purification column was cleaned with 5 mL of 20% alcohol;
(2) 1.5 mL of fillers were added into the purification column, and the purification column filled with the filler was cleaned with 3 column volumes of ultrapure water to remove alcohol;
(3) The purification column filled with filler was equilibrated with 2 column volumes of protein purification A solution (20 mM imidazole);
(4) A protein solution was allowed to pass through the column, for 5 times.
(5) The purification column filled with filler was equilibrated with 50 mL protein purification A solution (20 mM imidazole) per column;
(6) The column was repeatedly passed through with 30 mL washing liquid (50 mM imidazole) per column, for 3 times;
(7) The column was repeatedly passed through with 30 mL washing liquid (100 mM imidazole) per column, for 3 times;
(8) An interest protein was eluted by repeatedly passing 5 mL eluent (500 mM imidazole) through the column for 3 times;
(9) The purification column was cleaned with 20 mL eluent (500 mM imidazole);
(10) The purification column was cleaned with 20 mL pure water; and
(11) The purification column was cleaned with 15 mL 20% alcohol.

### 5. Animal Immunization and IL-17 Detection

10 experimental calves were randomly divided into 2 groups, wherein, 5 calves were injected subcutaneously with Brucellosis live vaccine A19 (6.0×10¹⁰ CFUs per calf), and 5 calves were injected with physiological saline as control. 30 days after immunization, the anti-coagulated blood samples of all the experimental calves were collected from veins at root of tails thereof (2 ml/tube), to each sample, 100 ml of Brucellosis cell immune protein at a concentration of 1000 μg/ml was added, the resultant was incubated at 37° C. for 24 h, a supernatant was collected by centrifugation, and the IL-17 level in the supernatant was detected by an ELISA method.

The specific ELISA method was as follows:
(1) The required plate strips were taken out after being equilibrated at room temperature for 20 min;
(2) Standard product wells and sample wells were arranged, and 50 μL of standard products at different concentrations were added into individual standard product wells;
(3) 50 μL of sample to be tested was added into the sample well, but not into blank well;
(4) 100 μL of horseradish peroxidase (HRP)-labeled detection antibody was added to each of the standard product wells and the sample wells except for the blank wells, and the reaction wells were sealed by a plate-sealing film, and incubated in an incubator at 37° C. for 60 min;
(5) The liquid was discarded, and the resultant was dried by flicking on an absorbent paper, and each well was fully filled with washing liquid (350 μL), and allowed to stand for 1 min.
(6) The plate was repeatedly washed with PBST, and dried by flicking on an absorbent paper, for 5 times;
(7) 50 μL of substrate A and 50 μL of substrate B were added to each well, and the resultant was incubated away from light at 37° C. for 15 min; and
(8) 50 μL of stop solution was added to each well, and an OD value was measured at a wavelength of 450 nm within 15 min.

All the antigens and the corresponding concentrations of IL-17 induced by them were shown in Table 3.

TABLE 3
IL-17 detection results after incubation
of antigen in peripheral blood

| Antigen | IL-17 Concentration (pg/ml) |
|---|---|
| BMEI0845* | 46.119 ± 3.414 |
| BMEI0178* | 45.339 ± 5.380 |
| BMEI1536* | 50.972 ± 2.388 |

### Example 2. Optimal Protein Concentration and Incubation Time Screening

According to the parameters and methods in Example 1, the working concentrations and treatment times of the three Brucellosis cell immune proteins BMEI0845*, BMEI0178* and BMEI1536* were adjusted to detect IL-17 concentration, and the specific experimental grouping and detection results were shown in Tables 4-6.

TABLE 4
Detection results of IL-17 at different concentrations
of BMEI0845* for different treatment times

| Antigen | | | | | |
|---|---|---|---|---|---|
| protein | | | | | |
| concentration | IL-17 concentration (pg/ml) | | | | |
| (μg/ml) | 16 h | 24 h | 32 h | 40 h | 48 h |
| 600 | 13.861 | 38.524 | 69.046 | 59.228 | 55.204 |
| 800 | 14.145 | 43.742 | 70.572 | 82.894 | 77.552 |
| 1000 | 30.958 | 97.465 | 124.264 | 102.344 | 76.859 |
| 1200 | 24.659 | 108.999 | 128.955 | 116.734 | 72.256 |
| 1500 | 21.383 | 95.976 | 116.732 | 99.223 | 99.1 |

TABLE 5
Detection results of IL-17 at different concentrations
of BMEI0178* for different treatment times

| Antigen | | | | | |
|---|---|---|---|---|---|
| protein | | | | | |
| concentration | IL-17 concentration (pg/ml) | | | | |
| (μg/ml) | 16 h | 24 h | 32 h | 40 h | 48 h |
| 600 | 32.064 | 67.242 | 85.852 | 91.801 | 87.98 |
| 800 | 11.054 | 35.919 | 50.433 | 117.464 | 108.247 |
| 1000 | 14.038 | 82.942 | 93.032 | 146.181 | 111.556 |
| 1200 | 30.295 | 37.533 | 97.134 | 120.796 | 103.308 |
| 1500 | 43.209 | 56.3 | 77.483 | 99.148 | 82.942 |

TABLE 6
Detection results of IL-17 at different concentrations
of BMEI1536* for different treatment times

| Antigen | | | | | |
|---|---|---|---|---|---|
| protein | | | | | |
| concentration | IL-17 concentration (pg/ml) | | | | |
| (μg/ml) | 16 h | 24 h | 32 h | 40 h | 48 h |
| 600 | 34.674 | 57.431 | 64.914 | 94.677 | 76.831 |
| 800 | 34.85 | 50.038 | 110.192 | 134.624 | 91.105 |
| 1000 | 56.067 | 95.234 | 165.777 | 174.898 | 157.113 |
| 1200 | 32.064 | 90.615 | 101.565 | 117.914 | 91.029 |
| 1500 | 75.976 | 74.412 | 86.936 | 95.234 | 90.615 |

It can be seen from Tables 4-6 that, an optimal concentration of the antigen protein BMEI0845* was 1000-1500 μg/ml, an optimal incubation time thereof was 24-40 h, and at this time, the concentration of IL-17 may reach 95.976-128.955 pg/ml; an optimal concentration of the antigen protein BMEI0178* was 800-1200 μg/ml, and an optimal incubation time thereof was 40-48 h, and at this time, the concentration of IL-17 may reach 103.308-146.181 pg/ml; and an optimal concentration of the antigen protein BMEI1536* was 800-1200 μg/ml, an optimal incubation time thereof was 32-48 hours, and at this time, the concentration of IL-17 may reach 91.029-174.898 pg/ml. Among the three antigen proteins, the BMEI1536* had the best immunogenicity, and at this time, the concentration of IL-17 produced by the BMEI1536* incubation with the whole blood of the experimental calf after immunization may reach 174.898 pg/ml.

### Example 3. Detection of IL-17 Level after Immunization Challenge

According to the parameters and methods in Example 1, experimental calves were subjected to immunization; 30 days after immunization, the calves were challenged with Brucella ovis M28 bacteria solution, and 40 days after challenge, the calves were slaughtered; and the optimal conditions of BMEI1536* protein were used to detect the bacteria loading capacity in the tissues and the concentrations of IL-17 at different immunization times. The specific experimental results were detailed in Tables 7 and 8.

The preparation steps of challenge bacteria solution were as follows: the Brucella ovis M28 bacteria strains were streak-inoculated in a TSA culture medium, and cultured at 37° C. for 48 h, then single colonies were picked and streaked in a TSA dish, and cultured at 37° C. for 72 h. To the dish, a TSB culture medium was added, the colonies were washed off after being soaked for 5 minutes, and the bacteria solution was transferred into a 50 ml centrifuge tube, to which a sterile glycerol solution was added to allow the final concentration thereof to be 20% v/v, and the resultant was uniformly mixed and counted, and then stored in a refrigerator at −20° C. to be ready for use. The counted dish was placed at 37° C., and cultured for 72 h, and according to the counting results, the bacteria solution of the Brucella ovis M28 strains was adjusted with sterile PBS to be at 1×10⁹ CFUs/ml.

TABLE 7
Bacteria loading capacity (CFU) in the tissues and protection results
(%) after immunization challenge of the experimental calves

| | | | Submandibular | Inguinal | Protection |
|---|---|---|---|---|---|
| Group | No. | Spleen | lymph node | lymph node | rate (%) |
| Vaccine | 971 | 0 | 0 | 0 | 80 |
| group | 972 | 0 | 2 | 1 | |
| | 973 | 0 | 0 | 0 | |
| | 974 | 0 | 0 | 0 | |
| | 975 | | 0 | 0 | |
| Control | 976 | 14 | 0 | 3 | 0 |
| group | 977 | 0 | 5 | 34 | |
| | 978 | 41 | 24 | 1 | |
| | 979 | 7 | 19 | 1 | |
| | 980 | 11 | 3 | 3 | |

TABLE 8
Detection results of IL-17 at different time points
after immunization of the experimental calves

| | | IL-17 (pg/ml) | | | |
|---|---|---|---|---|---|
| | | Before | | | 40 days |
| | | immuni- | 14 days after | 30 days after | after |
| Group | No. | zation | immunization | immunization | challenge |
| Vaccine | 971 | 13.313 | 52.978 | 117.144 | 181.02 |
| group | 972 | 14.455 | 38.905 | 38.365 | 43.68 |
| | 973 | 15.649 | 59.356 | 115.502 | 148.567 |
| | 974 | 16.267 | 64.878 | 132.537 | 185.511 |
| | 975 | 18.24 | 66.337 | 141.134 | 193.479 |
| Control | 976 | 12.237 | 13.195 | 18.199 | 27.144 |
| group | 977 | 13.283 | 18.053 | 16.011 | 24.365 |
| | 978 | 23.694 | 14.807 | 19.924 | 25.502 |
| | 979 | 13.739 | 16.713 | 18.524 | 22.537 |
| | 980 | 14.175 | 10.426 | 16.16 | 31.134 |

It can be seen from Tables 7-8 that, the results of challenge protection after immunization with a Brucella vaccine were assessed based on bacteria loading capacity in the tissues, 80% of the calves got protection, no corresponding challenging strains were isolated from the tissues thereof, and the protection results of the corresponding calves were consistent with the concentrations of IL-17 in trend. Calves (971, 973, 974, 975) with the IL-17 concentrations at a high level got protection, and no challenging strains were isolated; and when no immunization was carried out (976, 977, 978, 979, 980) or the immune level was insufficient (972), and when the IL-17 concentrations were at a low level, the calves got no protection, and the challenging strains were isolated from the tissues thereof, and the calves were at an infectious state.

The above results showed that, the level of IL-17 concentration can reflect the immunization state of the animal body after immunization with a Brucella vaccine. The Brucellosis cell immune protein was prepared as a reagent for detecting the immune level of the Brucella vaccine, and the reagent assesses the efficacy of vaccine immunization by detecting IL-17. Compared with traditional methods, the invention has relatively higher safety and simplicity.

The present invention provides concepts and processes of the Brucellosis cell immune protein and use thereof. There are many processes and routes for specific implementation of the technical solutions, and the above descriptions only illustrate preferred embodiments of the present invention. It should be noted that, those skilled in the art may make several improvements and modifications without departing from the principle of the present invention, and these improvements and modifications should also be regarded to be encompassed in the protection scope of the present invention. All the components that are not explicitly defined in the embodiments may be implemented by using the prior art.

1. A Brucellosis cell immune protein, characterized in that, the Brucellosis cell immune protein is any one of three antigens: BMEI1536*, BMEI0845* and BMEI0178*, wherein,
the BMEI1536* has a nucleotide sequence as shown in SEQ ID No.1;
the BMEI0845* has a nucleotide sequence as shown in SEQ ID No.2;
the BMEI0178* has a nucleotide sequence as shown in SEQ ID No.3.

2. The Brucellosis cell immune protein of claim 1, characterized in that, the Brucellosis cell immune protein is BMEI1536*.

3. The Brucellosis cell immune protein of claim 1, characterized in that, the Brucellosis cell immune protein is obtained by expressing a fusion protein sequence of a Brucella T cell epitope peptide fragment with antigen gene sequences of BMEI1536, BMEI0845 and BMEI0178 respectively, followed by recombinant transformation, incubation expression, and purification.

4. The Brucellosis cell immune protein of claim 3, characterized in that, the Brucella T cell epitope peptide fragment sequence is APGEKDGKIVPA, SEQ ID NO. 11, and has a nucleotide sequence as shown in SEQ ID No. 4.

5. The Brucellosis cell immune protein of claim 3, characterized in that, the recombinant transformation comprising synthesizing the sequence which is referred as to the fusion protein sequence into an expression vector pET-28a to obtain a recombinant plasmid, and then transforming the recombinant plasmid into a host strain BL21.

6. A reagent for detecting an immune level of a Brucella vaccine, characterized in that, the reagent comprises the Brucellosis cell immune protein of claim 1.

7. A method of detecting an immune level of a Brucella vaccine, characterized in that, the method uses the Brucellosis cell immune protein of claim 1.

8. The reagent of claim 6, characterized in that, the reagent is any one of BMEI1536*, BMEI0845* and BMEI0178*, preferably BMEI1536*.

9. The method of claim 7, characterized in that, specific detection steps comprise: collecting a blood sample of a calf after immunization, adding the Brucellosis cell immune protein thereto, collecting a supernatant after incubation, and detecting a concentration of IL-17 in the supernatant by an ELISA method.

10. The method of claim 9, characterized in that, the Brucellosis cell immune protein has a concentration of 600-1500 μg/ml; and the incubation is performed under the following conditions: at 37° C. for 16-48 h.

11. The reagent of claim 6, characterized in that, the Brucellosis cell immune protein is BMEI1536*.

12. The reagent of claim 6, characterized in that, the Brucellosis cell immune protein is obtained by expressing a fusion protein sequence of a Brucella T cell epitope peptide fragment with antigen gene sequences of BMEI1536, BMEI0845 and BMEI0178 respectively, followed by recombinant transformation, incubation expression, and purification.

13. The reagent of claim 12, characterized in that, the Brucella T cell epitope peptide fragment sequence is APGEKDGKIVPA, SEQ ID NO. 11, and has a nucleotide sequence as shown in SEQ ID No. 4.

14. The reagent of claim 12, characterized in that, the recombinant transformation comprising synthesizing the sequence which is referred as to the fusion protein sequence into an expression vector pET-28a to obtain a recombinant plasmid, and then transforming the recombinant plasmid into a host strain BL21.

15. The method of claim 7, characterized in that, the Brucellosis cell immune protein is BMEI1536*.

16. The method of claim 7, characterized in that, the Brucellosis cell immune protein is obtained by expressing a fusion protein sequence of a Brucella T cell epitope peptide fragment with antigen gene sequences of BMEI1536, BMEI0845 and BMEI0178 respectively, followed by recombinant transformation, incubation expression, and purification.

17. The method of claim 16, characterized in that, the Brucella T cell epitope peptide fragment sequence is APGEKDGKIVPA, SEQ ID NO. 11, and has a nucleotide sequence as shown in SEQ ID No. 4.

18. The method of claim 16, characterized in that, the recombinant transformation comprising synthesizing the sequence which is referred as to the fusion protein sequence into an expression vector pET-28a to obtain a recombinant plasmid, and then transforming the recombinant plasmid into a host strain BL21.
